(19) United States
(12) Patent Application Publication
(10) Pub. No.: US 2008/0021238 A1
(43) Pub. Date: Jan. 24, 2008

(54) Apparatus For (Meth) Acrylic Acid Production And Process For Producing (Meth) Acrylic Acid

(76) Inventors: Masahiko Yamagishi, Yokkaichi-shi (JP); Shuhei Yada, Tokyo (JP); Kimikatsu Jinno, Yokkaichi-shi (JP)

Correspondence Address: OBLON, SPIVAK, MCCLELLAND MAIER & NEUSTADT, P.C., 1940 DUKE STREET, ALEXANDRIA, VA, 22314, US

(21) Appl. No.: 11/569,578

(22) PCT Filed: Sep. 7, 2004
(86) PCT No.: PCT/JP04/12980

§ 371 Date: Nov. 24, 2006

(30) Foreign Application Priority Data
May 31, 2004 (JP) 2004-161257

(51) Int. Cl. C07C 51/00; B01J 19/00; F28D 21/00

(52) U.S. Cl. 562/512; 422/198

(57) ABSTRACT

(Meth)acrylic acid is produced using a reactor (1) through a vapor-phase catalytic oxidation reaction of propane or the like in a raw material gas, and the obtained reaction gas is distributed to a heat exchanger (20) and an absorption tower (30). Heat energy is recovered from the reaction gas supplied to the heat exchanger (20), and the reaction gas cooled in the heat exchanger (20) and the reaction gas distributed to the absorption tower (30) are supplied to the absorption tower (30). (Meth) acrylic acid is recovered from the reaction gas in an absorbing liquid, to thereby produce (meth) acrylic acid. The reaction gas is distributed to the heat exchanger (20) and the absorption tower (30) according to a pressure of the raw material gas at an inlet of the reactor (1). The present invention allows heat recovery from the reaction gas and a stable and continuous operation even when the heat exchanger for heat recovery is clogged.

### TECHNICAL FIELD

The present invention relates to an apparatus for and a method of producing (meth)acrylic acid through a vapor-phase catalytic oxidation reaction of propane, propylene, isobutylene, or (meth)acrolein. The present invention more specifically relates to an apparatus and a method for producing (meth)acrylic acid for preventing reduction in production of (meth)acrylic acid due to clogging of a heat exchanger provided between a reactor and an absorption tower, in recovering (meth)acrylic acid from a reaction gas discharged from the reactor in the absorption tower.

### BACKGROUND ART

A process for producing (meth)acrylic acid usually employs a method involving: producing (meth)acrylic acid through a vapor-phase catalytic reaction of propane, propylene, isobutylene, or (methacrolein; supplying a reaction gas containing the produced (meth)acrylic acid to an absorption tower to bring the reaction gas into contact with an absorbing liquid such as water; and recovering (meth)acrylic acid in the reaction gas as a (meth)acrylic acid solution.

Such a production process employs: a reactor receiving a catalyst for a vapor-phase catalytic oxidation reaction into which a raw material gas is introduced; and an absorption tower. A temperature of a reaction gas discharged from the reactor at this time is usually 250 to 350° C. Meanwhile, an absorption tower for (meth)acrylic acid is operated at a temperature of about 50 to 150° C. Thus, a process for producing (meth)acrylic acid generally employs an apparatus provided with a heat exchanger at an inlet of an absorption tower to cool a reaction gas, for purposes of recovering heat energy from the reaction gas, improving absorption efficiency of (meth)acrylic acid in the absorption tower, and the like (see JP 50-095217 A, JP 46-040609 B, and JP 08-176062 A, for example).

The reaction gas contains compounds such as phthalic acid and maleic acid in this case, and those compounds adhere to the heat exchanger during a continuous operation, leading to clogging of the heat exchanger. When the heat exchanger is clogged, a pressure in the reactor increases, developing difficulties in continuing a usual operation. In that case, an operation may be continued with reduced production of (meth)acrylic acid or an operation must be stopped for cleaning of the heat exchanger. Such clogging of the heat exchanger develops difficulties in a stable operation of a production apparatus for producing (meth)acrylic acid and reduces productivity of (meth)acrylic acid.

Examples of a known technique for removing a compound adhered to a heat exchanger include an apparatus having: a high boiling point impurities depositing zone provided in a reaction gas passages for absorbing high boiling point impurities in a reaction gas; and another high boiling point impurities depositing zone provided in the reaction gas passage which can be cleaned in a chamber adjacent to the reaction gas passage, to thereby remove high boiling point impurities from the reaction gas using the high boiling point impurities depositing zones (see JP 08-134012 A, for example).

Examples of a known technique for preventing formation of a deposit in a heat exchanger include a method involving: maintaining a cooling surface of the heat exchanger at a boiling point of maleic anhydride or more; and setting an average flow rate of a reaction gas at a predetermined rate or more (see JP 50-126605 A, for example).

However, nothing is described regarding adherence of a deposit to a heat exchanger in an apparatus provided with the heat exchanger for cooling a reaction gas supplied to an absorption tower. Thus, more consideration is needed on a stable operation of an apparatus for producing (meth)acrylic acid when such a deposit is adhered.

Furthermore the technique for removing a deposit in a heat exchanger or the technique for preventing adherence of a deposit to a heat exchanger may require a large scale apparatus for producing (meth)acrylic acid or complicated processes or may result in limited cooling of a reaction gas in the heat exchanger. Nothing is described on measures to clogging of the heat exchanger, and more consideration is needed on a stable operation of an apparatus for producing (meth)acrylic acid when a deposit is adhered to the heat exchanger.

### DISCLOSURE OF THE INVENTION

Accordingly, an object of the present invention is to provide a method which eliminates disadvantages of the prior art that is, a method which allows heat recovery from a reaction gas when (meth)acrylic acid in the reaction gas discharged from a reactor is supplied to an absorption tower to be recovered as a (meth)acrylic acid solution, and enables a stable and continuous operation even when a heat exchanger is clogged.

According to the present invention, in recovering acrylic acid or methacrylic acid hereinafter each or both of acrylic acid and methacrylic acid are collectively described as “(meth)acrylic acid”) as a (meth)acrylic acid solution by cooling a reaction gas discharged from a reactor using a heat exchanger and supplying the cooled reaction gas to an absorption tower, the heat exchanger for cooling the reaction gas is provided with a by-pass tube which connects an inlet and an outlet of the heat exchanger, and an inner pressure of the reactor is maintained at a predetermined value to prevent reduction in production of (meth)acrylic acid due to reduction in flow rate of a raw material gas to the reactor by gradually opening a valve provided in the by-pass tube when a pressure in the reactor increases and production of (meth)acrylic acid decreases due to clogging of the heat exchanger.

That is, the present invention provides an apparatus for producing (meth)acrylic acid comprising: a reactor for producing (meth)acrylic acid through a vapor-phase catalytic oxidation reaction of one or two or more of propane, propylene, isobutylene, and (meth)acrolein in a raw material gas containing one or two or more of propane, propylene, isobutylene, and (meth)acrolein, and oxygen; a heat exchanger for cooling a reaction gas comprising the produced (meth) acrylic acid; and an absorption tower for contacting an absorbing liquid for absorbing (meth)acrylic acid and the reaction gas so that the (meth)acrylic acid in the reaction gas is absorbed into the absorbing liquid, the apparatus for producing (meth)acrylic acid further including: a by-pass tube for connecting the reactor and the absorption tower without interposition by the heat exchanger; and a flow rate adjusting device for adjusting a flow rate of the reaction gas flowing through the by-pass tube.

The present invention further provides a method for producing (meth)acrylic acid by recovering (meth)acrylic acid absorbed in an absorbing liquid, including the steps of: generating (meth)acrylic acid using a reactor through a vapor-phase catalytic oxidation reaction of one or two or more of propane, propylene, isobutylene, and (meth)acrolein in a raw material gas containing one or two or more of propane, propylene, isobutylene, and (meth)acrolein, and oxygen; distributing a reaction gas containing the generated (meth)acrylic acid to a heat exchanger for cooling the reaction gas and to an absorption tower for contacting the reaction gas and the absorbing liquid for absorbing (meth)acrylic acid, cooling the reaction gas supplied to the heat exchanger using the heat exchanger; and contacting the reaction gas cooled in the heat exchanger and the reaction gas distributed to the absorption tower in the distribution step in the absorption tower so that (meth)acrylic acid in the reaction gas is absorbed into the absorbing liquid, wherein the reaction gas is distributed according to a flow rate of the raw material gas to the reactor in the distribution step.

### BRIEF DESCRIPTION OF THE DRAWINGS

FIG. 1 is a schematic diagram showing a structure of a production apparatus according to an embodiment of the present invention.

FIG. 2 is a diagram showing an embodiment of a multitube heat exchanger-type reactor used in a vapor-phase catalytic oxidation method according to the present invention.

FIG. 3 is a diagram showing an embodiment of a multitube heat exchanger-type reactor used in a vapor-phase catalytic oxidation method according to the present invention.

### BEST MODE FOR CARRYING OUT THE INVENTION

Industrially, (meth)acrolein or (meth)acrylic acid is generally obtained by oxidizing propane, propylene, isobutylene, and/or acrolein by molecular oxygen in the presence of a solid catalyst, that is, through so-called vapor-phase catalytic oxidation.

Hereinafter, examples of a process for producing (meth)acrylic acid will be explained taking acrylic acid for instance. The examples include the following (1) to (3).

(1) A process including: a step of producing acrylic acid through vapor-phase catalytic oxidation of propane, propylene, and/or acrolein; a collecting step of collecting acrylic acid as an aqueous solution of acrylic acid by bringing a gas containing acrylic acid produced in the step of producing acrylic acid into contact with water as an absorbing liquid; an extraction step of extracting acrylic acid from the aqueous solution of acrylic acid by using an appropriate extraction solvent; a step of separating the acrylic acid and the extraction solvent; a purification step of purifying the obtained acrylic acid; a step of recovering acrylic acid by decomposing a high boiling point liquid containing Michael adducts of acrylic acid and a polymerization inhibitor obtained from the above-mentioned steps; and a step of supplying acrylic acid to any of the steps after the collecting step.

(2) A process including: a step of producing acrylic acid through vapor-phase catalytic oxidation of propane, propylene, and/or acrolein; a collecting step of collecting acrylic acid as an aqueous solution of acrylic acid by bringing a gas containing acrylic acid produced in the step of producing acrylic acid into contact with water as an absorbing liquid; an azeotropic separation step of taking out crude acrylic acid from a bottom of an azeotropic separation tower by distilling the aqueous solution of acrylic acid in the presence of an azeotropic solvent; an acetic acid separation step of removing acetic acid from the obtained crude acrylic acid; a purification step of purifying the obtained acrylic acid; a step of recovering acrylic acid by decomposing a high boiling point liquid containing Michael adducts of acrylic acid and a polymerization inhibitor obtained from the above-mentioned steps; and a step of supplying acrylic acid to any of the steps after the collecting step.

(3) A process including: a step of producing acrylic acid through vapor-phase catalytic oxidation of propane, propylene, and/or acrolein; a collecting/separation step of collecting acrylic acid as an organic solution of acrylic acid by bringing a gas containing acrylic acid produced in the step of producing acrylic acid into contact with an organic solvent and simultaneously removing water, acetic acid, and the like; a separation step of taking out the acrylic acid from the organic solution of acrylic acid; a step of recovering acrylic acid by decomposing a high boiling point liquid containing Michael adducts of acrylic acid and a polymerization inhibitor obtained from the above-mentioned steps; a step of supplying acrylic acid to any of the steps after the collecting step; and a step of purifying part or whole of the organic solvent.

The present invention may employ any method of producing (meth)acrylic acid through the vapor-phase catalytic oxidation reaction without particular limitation.

The method for producing (meth)acrylic acid of the present invention includes the steps of: generating (meth)acrylic acid through a vapor-phase catalytic oxidation reaction of one or two or more of propane, propylene, isobutylene, and (meth)acrolein in a raw material gas containing one or two or more of propane, propylene, isobutylene, and (meth)acrolein, and oxygen using a reactor; distributing a reaction gas containing the generated (meth)acrylic acid to a heat exchanger for cooling the reaction gas and to an absorption tower for contacting the reaction gas an absorbing liquid for absorbing (meth)acrylic acid; cooling the reaction gas supplied to the heat exchanger using the heat exchanger; and contacting the reaction gas cooled in the heat exchanger and the reaction gas distributed to the absorption tower in the distribution step in the absorption tower so that (meth)acrylic acid in the reaction gas is absorbed into the absorbing liquid.

In the present invention, the steps of generating the (meth)acrylic acid, cooling the reaction gas using the heat exchanger, and absorbing the (meth)acrylic acid in the absorbing liquid can be carried out using known means such as a known apparatus or member.

In the present invention, the step of distributing a reaction gas involves distribution of the reaction gas generated in the step of generating (meth)acrylic acid to the heat exchanger and the absorption tower. The distribution is carried out according to the flow rate of the raw material gas to the reactor from the viewpoint of preventing reduction in flow rate of the raw material gas to the reactor.

When the raw material gas is supplied to the reactor utilizing a differential pressure between an inner pressure of the reactor and a pressure of the raw material gas, the distribution step is carried out according to a pressure of the raw material gas which is supplied to the reactor at an inlet of the reactor from the viewpoint of preventing reduction in flow rate of the raw material gas to the reactor due to increase of a pressure inside the reactor to reach a pressure identical to the pressure of the raw material gas supplied to the reactor.

In the distribution step, a distribution ratio of the reaction gas to the heat exchanger and to the absorption tower is not particularly limited so long as a desired flow rate of the raw material gas to the reactor can be secured. For example, the reaction gas produced in the reactor may be supplied to the heat-exchanger alone.

In the distribution step, the reaction gas is preferably distributed to provide a substantially constant flow rate of the raw material gas to the reactor from the viewpoint of stable production of (meth)acrylic acid. The phrase “substantially constant” as used herein means that the flow rate of the raw material gas to the reactor falls within a range not affecting the production of (meth)acrylic acid. Such a range differs depending on a scale of an apparatus or the like, however it is about ±5 vol % of the flow rate of the raw material gas to the reactor at the start of an operation of the production apparatus.

When the raw material gas is supplied to the reactor by utilizing a differential pressure between an inner pressure of the reactor and a pressure of the raw material gas, the reaction gas is preferably distributed to provide a substantially constant pressure of the raw material gas at an inlet of the reactor in the distribution step from the viewpoint of stable production of (meth)acrylic acid. The phrase “substantially constant” as used herein means that the pressure only needs to fall within a range depending on the above-mentioned numerical range of the flow rate of the raw material gas, and is about ±4 kPa with respect to the pressure of the raw material gas at an inlet of the reactor at the start of an operation of the production apparatus.

The distribution step can be carried out with a by-pass tube for diverting the reaction gas around the heat exchanger and a device for adjusting the flow rate of the reaction gas in the by-pass tube such as a valve. The flow rate of the reaction gas in the by-pass tube may be adjusted manually, however it is preferably adjusted by an automatic valve operating corresponding to a flowmeter for detecting a flow rate of the raw material gas to the reactor or a pressure gauge for detecting a pressure of the raw material gas at an inlet of the reactor.

The production method for (meth)acrylic acid of the present invention can be suitably carried out by using an apparatus for producing (meth)acrylic acid of the present invention described below.

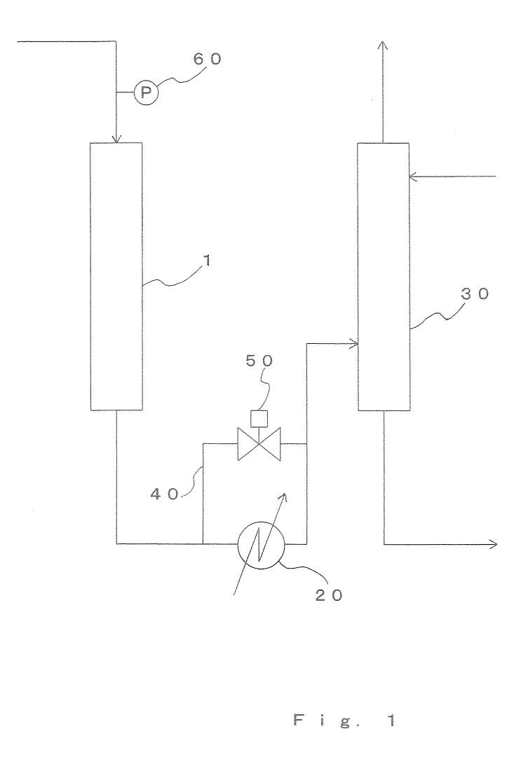

FIG. 1 shows an example of an apparatus for producing (meth)acrylic acid used in the present invention. The production apparatus is provided with: a reactor 1; a heat exchanger 20 for cooling a reaction product obtained in the reactor 1; an absorption tower 30 for absorbing in an absorbing liquid a predetermined component from the reaction product cooled in the heat exchanger 20; a by-pass tube 40 for connecting a tube from the heat exchanger 20 toward the reactor 1 and a tube from the heat exchanger 20 toward the absorption tower 30; and an automatic valve 50 for adjusting a flow rate of the reaction product flowing through the by-pass tube 40. The automatic valve 50 opens or closes according to a detected value of a pressure gauge 60 for detecting a pressure of the raw material gas at an inlet of the reactor 1 through which a raw material gas is supplied into the reactor 1. The production apparatus is optionally provided with not-shown apparatuses such as a rectifying tower and a decomposition reactor corresponding to the subsequent steps.

The reactor 1 is a device for generating (meth)acrylic acid through a vapor-phase catalytic oxidation reaction of one or two or more of propane, propylene, isobutylene, and (meth)acrolein in a raw material gas containing one or two or more of propane, propylene, isobutylene, and (meth)acrolein, and oxygen.

The present invention includes a method of producing acrylic acid through vapor-phase oxidation of propylene and/or acrolein by using molecular oxygen. Typical examples of a commercialized method of producing acrolein and acrylic acid through vapor-phase catalytic oxidation include a one-pass system, an unreacted propylene recycle system, and a flue gas recycle system described herein. A reaction system of the present invention is not limited so long as the system allows production of (meth)acrylic acid through a vapor-phase catalytic oxidation reaction including the three above-mentioned systems.

(1) One-pass System:

The one-pass system involves: mixing and supplying propylene airs and steam for a first reaction; converting the mixture to mainly acrolein and acrylic acid; and supplying an outlet gas for a second reaction without separating the products from the outlet gas. At this time, a general method also involves supplying air and steam required for a reaction in the second reaction to the second reaction in addition to the first reaction outlet gas.

(2) Unreacted Propylene Recycle System:

The unreacted propylene recycle system for recycling part of the unreacted propylene involves: guiding the reaction gas containing acrylic acid obtained in the second reaction to a collecting device for collecting acrylic acid; collecting the acrylic acid as an aqueous solution; and supplying part of a waste gas containing the unreacted propylene from the collecting device to the first reaction.

(3) Flue Gas Recycle System:

The flue gas recycle system involves: guiding the reaction product gas containing acrylic acid obtained in the second reaction to a collecting device for collecting acrylic acid; collecting the acrylic acid as an aqueous solution; combusting all waste gas from the collecting device; converting the unreacted propylene or the like in the waste gas to mainly carbon dioxide and water; and adding part of the obtained flue gas to the first reaction.

The reactor 1 is not particularly limited so long as it is a device allowing a reaction of an above-mentioned reaction system. An example of the reactor 1 includes a fixed bed multitube reactor. A vapor-phase catalytic oxidation reaction using the fixed bed multitube reactor is a method widely used in producing (meth)acrolein or (meth)acrylic acid from propane, propylene, or isobutylene in the presence of a mixed oxide catalyst using molecular oxygen or a molecular oxygen-containing gas.

The present invention employs a fixed bed multitube reactor generally used industrially without any particular limitation. Reactors of other types include a fixed bed plate reactor and a fluidized bed reactor which may also be employed as the reactor of the present invention.

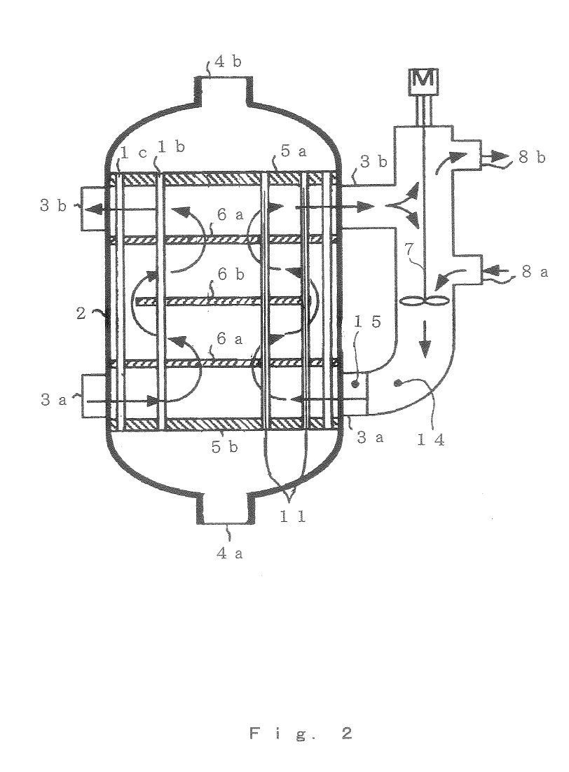

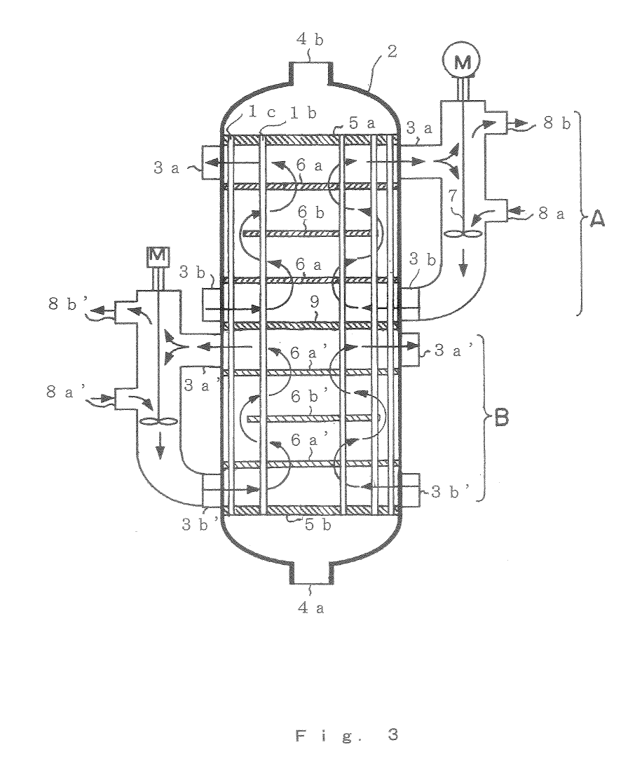

Hereinafter, a specific mode of the reactor will be described with reference to FIGS. 2 and 3.

As shown in FIGS. 2 the reactor 1 (hereinafter may also be referred to as “multitube reactor”) is provided with, for example: a shell 2; ports 4a and 4b formed on both ends of the shell 2, for serving as a raw material supply port which is an inlet of a raw material gas or as a product discharge port which is an outlet of a reaction gas containing the product; two tube plates 5a and 5b for dividing inside of the shell 2 in a transverse direction; a plurality of reaction tubes 1b and 1c passing through the tube plates 5a and 5b and fixed thereon; ring-shaped tubes 3a and 3b for circulating a heating medium between a space inside the shell 2 sandwiched by the two tube plates and outside of the shell 2; and perforated baffle boards 6a and 6b alternatively arranged in a longitudinal direction of the shell 2 in the space inside the shell 2 sandwiched by the two tube plates.

The reaction tubes 1b and 1c are packed with a catalyst or the like. Further, a thermometer 11 is inserted in each of the reaction tubes 1b and 1c. The catalyst or the like packed in the reaction tubes 1b and 1c will be described later.

The ring-shaped tubes 3a and 3b are provided with: a circulation pump 7 for circulating a heating medium between the ring-shaped tubes 3a and 3b and the shell 2; a heating medium supply line 8a for supplying the heating medium to the ring-shaped tubes 3a and 3b; a heating medium draw line 8b for drawing the heating medium from the ring-shaped tubes 3a and 3b; and a plurality of thermometers 14 and 15 for detecting a temperature of the heating medium.

The perforated baffle boards 6a and 6b are each provided to extend in a transverse direction of the shell 2 and are fixed on the reaction tubes 1b and 1c. The perforated baffle board 6a is, for example, a doughnut-shaped perforated baffle board extending from an inner peripheral wall to a vicinity of a central portion of the shell 2, thereby forming an opening portion in the vicinity of the central portion of the shell 2. The perforated baffle board 6b is, for example, a circular perforated baffle board extending from a central portion to an inner peripheral wall of the shell 2, thereby forming an opening between the inner peripheral wall of the shell 2 and an edge portion of the perforated baffle board 6b.

A shape or arrangement of each of the perforated baffle boards 6a and 6b are determined such that a projected image of all perforated baffle boards occupies a cross section of the shell 2 when all of the perforated baffle boards provided in the shell 2 are projected onto a cross section of the shell 2 from the viewpoint of preventing formation of hot spots (overheat portions) in the reaction tubes 1b and 1c.

In the reactor 1 shown in FIG. 2, as long as a process gas (raw material gas, reaction gas, or both thereof) and a heating medium are in a countercurrent flow, a flow direction of the process gas is not limited. In FIG. 2, the flow direction of he heating medium inside the shell 2 is indicated by arrows as an upflow, and thus reference numeral 4b represents the raw material supply port. The raw material gas introduced through the raw material supply port 4b successively reacts in the reaction tubes 1b and 1c of the reactor 1.

The heating medium pressurized with the circulation pump 7 flows upward inside the shell 2 from the ring-shaped tube 3a while absorbing heat of reaction generated through a vapor-phase catalytic oxidation reaction in the reaction tubes 1b and 1c. The flow direction of the heating medium introduced into the shell 2 is changed by alternatively arranging a plurality of the perforated baffle board 6a having an opening portion in the vicinity of the central portion of the shell 2 and the perforated baffle board 6b forming an opening portion in the vicinity of the inner peripheral wall of the shell 2. The heating medium is then returned to the circulation pump 7 through the ring-shaped tube 3b.

Part of the heating medium absorbing the heat of reaction flows through the heating medium draw line 8b provided in an upper portion of the circulation pump 7, is cooled with the heat exchanger (not shown), is introduced into the ring-shaped tube 3a again from the heating medium supply line 8a, and is introduced into the shelf 2 again. The heating medium temperature is adjusted by controlling a temperature or a flow rate of a returning heating medium introduced from the heating medium supply line 8a based on a temperature detected by a thermometer 14, for example.

The heating medium temperature is adjusted such that a temperature difference of the heating medium between the heating medium supply line 8a and the heating medium draw line 8b falls within 1° C. to 10° C., preferably 2° C. to 6° C., though depending on the performance of the catalyst used.

A current plate (not shown) is preferably provided in a shell plate portion inside each of the ring-shaped tubes 3a and 3b for minimizing a difference in flow rate of the heating medium flowing through a cross section including the shell plate portion. A porous plate or a plate provided with slits is used as the current plate, and an opening area of the porous plate or slit intervals is changed such that the heating medium flows into the shell 2 at the same flow rate from any position of the cross section. The temperature inside the ring-shaped tube (3a, preferably also 3b) can be monitored by providing a plurality of thermometers 15.

The number of the perforated baffle boards 6 provided inside the shell 2 is not particularly limited, however, three baffle boards (2 perforated baffle boards of 6a type and 1 perforated baffle board of 6b type) are preferably provided as usual. The perforated baffle boards 6 prevent a simple upflow of the heating medium, changes the flow of the heating medium to a lateral direction with respect to an axial direction of the reaction tubes. The heating medium converges from a peripheral wall portion to a central portion of the shell 2, changes direction in the opening portion of the perforated baffle board 6a, flows toward the peripheral wall portion of the shell 2, and reaches the peripheral wall of the shell 2.

The heating medium changes direction again on the peripheral wall by the perforated baffle board 6b, converges to the central portion of the shell 2, flows upward through the opening portion of the perforated baffle board 6a, flows along the tube plate 5a toward the peripheral wall of the shell 2 and returns to the circulation pump 7 through the ring-shaped tube 3b.

Thermometers 11 are inserted into the reaction tubes 1b and 1c provided inside the reactor 1 and signals are transmitted to the outside of the reactor 1, to thereby record temperature distributions of catalyst layers in an axial direction of the reactor 1. A plurality of thermometers are inserted into the reaction tubes 1, and one thermometer measures temperatures of 5 to 20 points in the reaction tubes 1b and 1c in an axial direction.

As the reactor 1, a reactor shown in FIG. 3 is employed, for example. A multitube reactor shown in FIG. 3 has the same structure as that of the multitube reactor shown in FIG. 2 except that the reactor is provided with an intermediate tube plate 9 for further dividing a space inside the shell 2 divided by the tube plates 5a and 5b; perforated baffle boards 6a and 6b in each of a space divided by the tube plate 5a and the intermediate tube plate 9 and a space divided by the intermediate tube plate 9 and the tube plate 5b; and ring-shaped tubes 3a and 3b for circulating the heating medium to each of a space divided by the tube plate 5a and the intermediate tube plate 9 and a space divided by the intermediate plate 9 and the tube plate 5b.

The spaces divided by the intermediate tube plate 9 in the shell 2 are controlled to different temperatures by supplying different heating media. A raw material gas may be introduced from either the port 4a or 4b. In FIG. 3 a flow direction of the heating medium inside the shell 2 is indicated by arrows as an upflow, and thus, reference numeral 4b represents the raw material supply port in which the process gas flows in a countercurrent flow to the heating medium. The raw material introduced from the raw material supply port 4b successively reacts inside the reaction tubes 1b and 1c of the reactor 1.

The multitube reactor shown in FIG. 3 may include heating media having different temperatures in a space divided by the tube plate 5a and the intermediate tube plate 9 (area A in FIG. 3) and in a space divided by the intermediate tube plate 9 and the tube plate 5b (area B in FIG. 3). Such a difference of temperature zones may be effectively used depending on packing specifications of the catalyst or the like in the reaction tubes.

Examples of such a case include: 1) a case where each reaction tube is entirely packed with the same catalyst and the temperature of the raw material gas is changed at an inlet and an outlet of the reaction tube for a reaction; 2) a case where an inlet portion of the raw material gas is packed with a catalyst and an outlet portion of the process gas is packed with no catalyst that is, left as a cavity or packed with an inert substance without reaction activity, for rapidly cooling a reaction product, and 3) a case where the inlet and outlet portions of the raw material gas are packed different catalysts and a space therebetween is packed with no catalyst, that is, left as a cavity or is packed with an inert substance for rapid cooling of a reaction product without reaction activity.

For example, a mixed pas containing propylene propane, or isobutylene and a molecular oxygen-containing gas is introduced into the multitube reactor shown in FIG. 3 from the raw material supply port 4b. First, the mixed gas is converted to (meth)acrolein in a first stage (area A of reaction tubes) for a first reaction, and the (meth)acrolein is then oxidized in a second stage (area B of reaction tubes) for a second reaction, to thereby produce (meth)acrylic acid.

A first stage portion of the reaction tubes (hereinafter may also be referred to as “first stage portion” and a second stage portion of the reaction tubes (hereinafter, may also be referred to as “second stage portion” are packed with different catalysts and are controlled to different temperatures for a reaction under optimum conditions. The inert substance not involved in the reaction is preferably packed between the first stage portion and the second stage portion of the reaction tubes (portion supported by the intermediate tube plate 9 and vicinity thereof).

In each of FIGS. 2 and 3, the flow direction of the heating medium in the shell 2 is represented by arrows as an upflow. However, the present invention can also be applied to the opposite flow direction. Regarding circulation of the heating medium, the heating medium is preferably circulated to prevent a phenomenon of entraining, with the heating medium, a gas, specifically, an inert gas such as nitrogen existing on upper ends of the shell 2 and the circulation pump 7 or realizing stable production of (meth)acrylic acid.

The heating medium draw line 8b is preferably provided at least above the tube plate 5a from the viewpoint of increasing a pressure inside the shell 2. Such a structure can prevent stagnation of a gas in he shell 2 or the ring-shaped tubes 3a and 3b and a cavitation phenomenon of the circulation pump 7. When a stagnation portion of the gas is formed above the shell 2, an upper portion of the reaction tubes provided in the gas stagnation portion may not be cooled by the heating medium, but such a structure can prevent insufficient temperature control of the heating medium.

In a multitube reactor oxidizing propylene, propane, or isobutylene with a molecular oxygen-containing gas and employing the multitube reactor shown in FIG. 2, when a process gas is a downflow, that is, when the raw material gas is introduced from the port 4b and the product is discharged from the port 4a, the target product, (meth)acrolein, has high concentration and is heated by the heat of reaction. Thus, the process gas temperature may also increase in the vicinity of the port 4a where the product is discharged.

Further, in a multitube reactor employing the multitube reactor shown in FIG. 3, when a process gas is a downflow, that is, when the raw material gas is introduced from the port 4b and the product is discharged from the port 4a, the target product, (meth)acrolein, has high concentration and is heated by the heat of reaction, and thus the process gas temperature may also increase in the vicinity of the intermediate tube plate 9 which is an end point of the first stage (area A of reaction tubes).

When the catalyst is packed in the first stage alone (area A of reaction tubes: 5a-6a-6b-6a-9), a reaction is inhibited in the second stage of the reaction tubes 1b and 1c (area B of reaction tubes: between 9 and 5b) and the process gas is cooled by the heating medium flowing through the reaction area B of the shell 2, to thereby prevent an autooxidation reaction of (meth)acrolein. In this case, area B of the reaction tubes 1b and 1c (between 9 and 5b) packed with no catalyst, which are left as cavities or packed with a solid without reaction activity. The latter is desirable for improving heat transfer characteristics.

Further, when different catalysts are lacked in the first stage (area A of reaction tubes: 5a-6a-6b-6a-9) and the second stage (area B of reaction tubes: 9-6a′-6b′-6a′-5b) of the multitube reactor shown in FIG. 3 for obtaining (meth)acrolein from propylene, propane, or isobutylene in the first stage and obtaining (meth)acrylic acid in the second stage, a catalyst layer temperature of the first stage may be higher compared to the catalyst layer temperature of the second stage. Specifically, the first stage (6a-9) near the end point of the reaction and the second stage (9-6a) near the starting point of the reaction have high temperatures.

Thus, it is preferable that reactions are not performed in those portions and the process gas is cooled by the heating medium flowing through the shell 2 in the vicinity of the intermediate tube plate 9, to thereby prevent an autooxidation reaction of (meth)acrolein. In this case, portions packed with no catalyst are provided in the vicinity of the intermediate tube plate 9 (portions within 6a-9-6a′ of reaction tubes 1b and 1c), which are left as cavities or packed with a solid without reaction activity. The latter is desirable for improving heat transfer characteristics.

Examples of the catalyst used for a vapor-phase catalytic oxidation reaction for producing (meth)acrylic acid or (meth)acrolein include: a catalyst used in the first reaction for producing unsaturated aldehyde or unsaturated acid from an olefin; and a catalyst used in the second reaction for producing unsaturated acid from unsaturated aldehyde. The present invention may employ either catalyst.

In the vapor-phase catalytic oxidation reaction, a Mo—Bi mixed oxide catalyst can be used in a first reaction (reaction for converting an olefin into unsaturated aldehyde or unsaturated acid) for producing mainly acrolein. Examples of the Mo—Bi mixed oxide catalyst include a compound represented by the general formula (I).
MoₐW_{b}Bi_{c}Fe_{d}AₑB_{f}C_{g}DₕEᵢOₓ (I)
(wherein, Mo represents molybdenum; W represents tungsten; Bi represents bismuth; Fe represents iron; A represents at least one element chosen from nickel and cobalt; B represents at least one element selected from the group consisting of sodium, potassium, rubidium, cesium, and thallium; C represents at least one element selected from alkali earth metals; D represents at least one element selected from the group consisting of phosphorus, tellurium, antimony, tin, cerium, lead, niobium, manganese, arsenic, boron, and zinc; E represents at least one element selected from the group consisting of silicon, aluminum, titanium, and zirconium; C represents oxygen; a, b, c, d, e, f, g, h, i, and x represent atomic ratios of Mo, W, Bi, Fe, A, B, C, D, E, and O respectively; and if a=12, 0≦b≦10, 0<c≦10 (preferably 0.1≦b≦10), 0<d≦10 (preferably 0.1≦d≦10), 2≦e≦15, 0<f≦10 (preferably 0.001≦f≦10), 0≦g≦10, 0≦h≦4, and 0≦i≦30; and x is a value determined from oxidation states of the respective elements.)

In the vapor-phase catalytic oxidation reaction, a Mo—V mixed oxide catalyst can be used in a second reaction (reaction for converting unsaturated aldehyde into unsaturated acid) for oxidizing acrolein to produce acrylic acid. Examples of the Mo—V mixed oxide catalyst include a compound represented by the general formula (II).
MoₐV_{b}W_{c}Cu_{d}XₑY_{f}O_{g} (II)
(wherein, Mo represents molybdenum; V represents vanadium; W represents tungsten; Cu represents copper; X represents at least one element selected from the group consisting of Mg, Ca, Sr, and Ba; Y represents at least one element selected from the group consisting of Ti, Zr, Ce, Cr, Mn, Fe, Co, Ni, Zn, Nb, Sn, Sb, Pb, and Bi; C represents oxygen; a, b, c, d, e, f, and g represent atomic ratios of Mo, V, W, Cu, X, Y, and O respectively; if a=12, 2≦b≦14, 0≦c≦12, 0<d≦6, 0≦e≦3, and 0≦f≦3; and g is a value determined from oxidation states of the respective elements.)

The above-mentioned catalysts may be produced through methods disclosed in JP 63-054942 A, JP 06-013096 B. H 06-038918 B. and the like.

A catalyst used in the present invention may be a molded catalyst molded through extrusion molding or tablet compression or may be a supported catalyst prepared by supporting a mixed oxide composed of a catalyst component on an inert support such as silicon carbide, alumina, zirconium oxide, or titanium oxide.

A shape of the catalyst used in the present invention is not particularly limited and may be spherical, columnar, cylindrical, star-shaped, ring-shaped, amorphous, or the like.

The above-mentioned catalysts may be used in combination with an inert substance as a diluent. The inert substance is not particularly limited so long as the inert substance is stable under the reaction conditions and has no reactivity to a raw material substance and a product. Specific examples of the inert substance include those used for catalyst supports such as alumina, silicon carbide, silica, zirconium oxide, and titanium oxide.

The shape of the inert substance, similar to that of the catalyst, is not limited and may be spherical columnar, cylindrical, star-shaped, ring-shaped, fragmented; meshed; amorphous, or the like. The size of the inert substance may be determined in consideration of a diameter of a reaction tube and a pressure difference.

An amount of the inert substance used as a diluent is determined arbitrarily depending on an expected catalyst activity.

Examples of a method of packing a catalyst and an inert substance corresponding to such purposes include: a method involving dividing a packed bed of a reaction tube, increasing the amount of the inlet substance used near a raw material gas inlet or the reaction tube for lowering the catalyst activity to suppress heat generation, and reducing the amount of the inert substance used near a reaction gas outlet of the reaction tube for enhancing the catalyst activity to accelerate the reaction; and a method involving packing the catalyst and the inert substance in the reaction tubes in one layer at a fixed mixing ratio.

Examples of a method of changing the catalytic activity in the reaction tube include: adjusting a catalyst composition to use a catalyst having a different catalytic activity; and mixing catalyst particles and inert substance particles for dilution of the catalyst to adjust the catalytic activity.

Specific examples of two layer packing involves: using a catalyst having a large ratio of inert substance particles, that is, containing the inert substance particles at a ratio of 0 3 to 0.7 with respect to total packing in an inlet portion of the raw material gas in the reaction tubes; and using a catalyst having a smaller ratio of the inert substance particles (inert substance particles at a ratio of 0.5 to 1.0 with respect to total packing, for example in an outlet portion of the reaction gas in the reaction tube.

The number of catalyst layers formed in an axial direction of the fixed bed multitube reactor is not particularly limited. However, too large a number of the catalyst layers requires extensive work in a catalyst packing process, and the number thereof is usually 1 to 10. A length of each the catalyst layers is arbitrarily determined depending on the catalyst type, the number of catalyst layers, the reaction conditions, or the like.

A mixed gas containing propylene, propane, isobutylene, and/or (meth)acrolein, a molecular oxygen-containing gas, and steam is mainly introduced as a raw material gas into the multitude reactor used in the vapor-phase catalytic oxidation.

In the present invention, a concentration of propylene, propane, or isobutylene in the raw material gas is 6 to 10 mol %. A concentration of oxygen is 1.5 to 2.5-mol times that of propylene, propane, or isobutylene, and a concentration of the steam is 0.8 to 5-mole times that of propylene, propane, or isobutylene. The introduced raw material gas is divided into the respective reaction tubes and passes through each of the reaction tubes, and reacts in the presence of an oxidation catalyst packed therein.

The heat exchanger 20 is not particularly limited so long as it is a device for cooling the reaction gas produced in the reactor 1. A heat exchanger of any type such as a multitude heat exchanger, a plate heat exchanger, or a spiral heat exchanger can be used as such a heat exchanger 20. A multitude heat exchanger, which allows easy cleaning of the heat exchanger when a high boiling point substance is adhered, can be particularly preferably used.

In this case, a reaction gas may flow through either a tube side or a shell side of heat exchanger 20. However, the reaction gas preferably flows through the tube side for reducing a pressure difference of the reaction gas and allowing easy cleaning of a deposit.

A flow rate of the reaction gas in the multitube heat exchanger is 5 to 25 m/sec., preferably 5 to 15 m/sec. Too small a flow rate undesirably tends to increase adherence of a high boiling point substance to the heat exchanger. Too large a flow rate undesirably tends to increase a pressure difference in the heat exchanger, to thereby increase a reaction pressure.

A temperature of a heating (cooling) medium of the heat exchanger 20 falls within a range of 100 to 250° C., preferably 120 to 200° C. Too low a temperature of the heating medium is disadvantageous because heat energy of the reaction gas cannot be recovered as steam. Too high a temperature of the heating medium is not preferable because recoverable heat energy decreases.

Examples of a method of cooling a reaction gas by the heating medium in the heat exchanger 20 include: cooling by using an organic heating medium; cooling by using pressurized water; and cooling by boiling water. The present invention may employ any method without problems.

The absorption tower 30 is a device for absorbing in an absorbing liquid (meth)acrylic acid in the reaction gas by bringing the absorbing liquid for absorbing (meth)acrylic acid into contact with the reaction gas. Such an absorption tower 30 may employ a tower provided with: a reaction gas supply port in a lower portion; an absorbing liquid supply port in an upper portion; packing or trays packed between the ports; and a liquid discharge port in a bottom portion.

Trays or packing is provided inside the absorption tower 30. Specific examples of trays include bubble cap trays each having a downcomer, perforated-plate trays, valve trays, SUPERFRAC trays, baffle trays, MAX-FRAC trays, and dual flow trays without downcomers.

Examples of packing include stacked packing and dumped packing. Examples of stacked packing include: SULZER PACKING available from Sulzer Brothers Ltd.; SUMITOMO-SULZER PACKING available from Sumitomo Heavy Industries, Ltd.; MELLAPAK available from Sumitomo Heavy Industries, Ltd; EM-PAK available from Koch-Glitsch, LP; MONTZ-PAK available from Julius Montz GmbH; GOOD ROLL PACKING available from Tokyo Tokushu Kanaami K. K.; HONECYOCM PACK available from NGK Insulators, Ltd.; IMPULSE PACKING available from Nagaoka International Corporation; and MC PACK available from Mitsubishi Chemical Engineering Corporation.

Examples of dumped packing include: INTALOX SADDLES available from Saint-Gobain NorPro; TELLERETT available from Nittetsu Chemical Engineering Ltd.; PALL RINGS available from BASF Aktiengesellschaft; CASCADE MINI-RING available Mass Transfer Ltd.; and FLEXI RINGS available from JGC Corporation.

The type of trays and packing is not limited in the present invention, and one or more types each of trays and packing can be used in combination as generally used.

The absorbing liquid is not particularly limited so long as the liquid absorbs (meth)acrylic acid from the reaction gas. Examples of such an absorbing liquid include water, an organic solvent such as diethyl terephthalate, and a mixture of water and an organic solvent.

A supply method for an absorbing liquid in the absorption tower 30 is not particularly limited so long as the method brings the reaction gas into contact with the absorbing liquid The present invention may employ any method without problems including: a method of supplying the absorbing liquid to be brought into contact with the reaction gas in a countercurrent flow; a method of bringing the reaction gas into contact in a concurrent flow with the absorbing liquid for absorption; and a method of bringing the reaction gas into contact with the absorbing liquid sprayed in advance, cooling the whole, and absorbing the reaction gas in the absorbing liquid.

The by-pass tube 40 is not particularly limited so long as it is a tube connecting the reactor 1 and the absorption tower 30 without interposition by the heating exchanger 20. The by-pass tube 40 may be provided directly in a main body of the heat exchanger 20 or may be provided on a tube connected to the heat exchanger 20. The by-pass tube 40 need not be one tube, and a plurality of by-pass tubes may also be used.

The automatic valve 50 is a device for adjusting a flow rate of the reaction gas flowing through the by-pass tube 40. The embodiment of the present invention employs the automatic valve 50, but the present invention may employ various means without particular limitation so long as the valve is a device capable of adjusting the flow rate of the reaction gas in the by-pass tube 40. Examples of a flow rate adjusting device that can be used without problems include: a valve capable of adjusting an opening automatically; and a valve capable of changing an opening manually as required.

Examples of a valve type include a globe valve, a needle valve, a gate valve, and a butterfly valve, but any valve may be used as long as the valve is capable of changing an opening of the valve.

Materials for various nozzles, a tower body, a reboiler, tubes, impingement plates (including a top plate), and the like as various components of a distillation tower used in the apparatus for producing (meth)acrylic acid in the present invention are selected depending on easily polymerizable compounds used such as (meth)acrylate, raw materials thereof, and intermediates and temperature conditions. However, the materials are not particularly limited in the present invention so long as the materials do not cause problems in processes of the present invention.

For example, stainless steels are often used as such materials in production of (meth)acrylic acid and (meth)acrylates, which are typical easily polymerizable substances and the present invention may employ such metals as materials. However, the materials are not limited to stainless steels. Examples of materials for various components include SUS 304, SUS 304 , SUS 316, SUS 316L, SUS 317, SUS 317L, SUS 327, and hastelloys. The materials for various components may be selected corresponding to physical properties of each liquid from the viewpoint of corrosion resistance or the like.

In the reactor 1, the above-mentioned raw material gas is supplied to the shell 2 from the port 4b and the raw material gas is supplied to the reaction tubes 1b and 1c packed with the above-mentioned catalyst, to thereby produce (meth)acrylic acid. The reaction gas containing the produced (meth)acrylic acid is discharged from the reactor 1 at 200 to 350° C.

The reaction gas discharged from the reactor 1 is supplied to the heat exchanger 20 and cooled, to thereby recover heat energy from the reaction gas. At an initial state, the automatic valve 50 may be closed completely.

The reaction gas cooled to 150 to 250° C. in the heat exchanger 20 is supplied to the absorption tower 30. The reaction gas supplied to the absorption tower 30 flows upward through the tower from a lower portion of the absorption tower 30, and is brought into contact with the absorbing liquid (water, for example) sprayed from an upper portion of the absorption tower 30. The reaction gas and the absorbing liquid are efficiently brought into contact with each other by trays or packing in the absorption tower 30, and (meth)acrylic acid in the reaction gas is absorbed in the absorbing liquid. An aqueous solution of (meth)acrylic acid obtained through contact thereof is received at a bottom of the absorption tower 30 and drawn from the absorption tower 30.

In the absorption tower 30, a gas component which is not absorbed in the absorbing liquid is discharged from a top of the absorption tower 30, and partially returned to the reactor 1 or supplied to a detoxification treatment facility for atmospheric discharge.

The aqueous solution of (meth)acrylic acid drawn from the absorption tower 30 is subjected to dehydration, separation of low boiling point components or the like through a conventionally known method to thereby recover purified acrylic acid from the aqueous solution of (meth)acrylic acid.

Meanwhile, the reaction gas discharged from the reactor 1 contains a high boiling point substance such as maleic anhydride, terephthalic acid, or trimellitic acid. Such a high boiling point substance adheres to the heat exchanger 20 to gradually increase a pressure difference of the heat exchanger 20. Thus, continuous production of (meth)acrylic acid gradually increases a pressure of the raw material gas at an inlet of the reactor 1 a pressure inside the reaction tubes of the reactor 1, and a pressure at an outlet of the reactor 1.

When the pressure of the raw material gas at an inlet of the reactor 1 increases to a level identical to a supply pressure of the reaction gas, the raw material gas is hardly supplied to the reactor 1. Thus, the flow rate of the raw material gas to the reactor 1 must be reduced for an operation with reduced production of (meth)acrylic acid or the operation must be stopped for cleaning the heat exchanger 20.

In the embodiment of the present invention, the automatic valve 50 opens the by-pass tube 40 according to a detection value of the pressure gauge 60, to thereby maintain a pressure of the raw material gas at an inlet of the reactor 1 at a constant value. Thus, a pressure of the raw material gas at an inlet of the reactor 1 reduces, and production of (meth)acrylic acid may be continued without changing the flow rate of the raw material gas to the reactor 1.

The automatic valve 50 may continuously adjust an opening of the valve or an operator may change an opening occasionally as required to provide a constant pressure of the reactor 1 or a constant flow rate of the raw material gas to the reactor 1.

The automatic valve 50 is preferably closed completely at the start of the operation from the viewpoint of increasing recovery of heat energy from the reaction gas However, the automatic valve 50 may be opened immediately after the start of the operation from the viewpoints of preventing clogging of the heat exchanger 20 and adjusting the temperature of the reaction gas.

More specifically examples of a method of adjusting a pressure of the raw material gas at an inlet of the reactor 1 include a method involving carrying out an operation with the automatic valve 50 opened at a fixed opening from the start of the operation and gradually opening the automatic valve 50 when a pressure of the raw material gas at an inlet of the reactor 1 increases with adherence of a high boiling point substance, to thereby maintain a constant pressure of the raw material gas at an inlet of the reactor 1; and a method involving gradually opening the automatic valve 50 when a pressure of the raw material gas at an inlet of the reactor 1 reaches a level identical to the pressure of the reaction gas supplied to the reactor 1 to develop difficulties in supply of the raw material gas and to inhibit secure production of (meth)acrylic acid, to thereby adjust the pressure of the raw material gas at an inlet of the reactor 1. Such a method is preferable from the viewpoint of maintaining constant production of (meth)acrylic acid.

In the embodiment of the present invention, a pressure of the raw material gas at an inlet of the reactor 1 is detected by the pressure gauge 60 to adjust the opening and closing of the automatic valve 50. However, the position and number of the pressure gauge 60 is not particularly limited so long as the pressure gauge can detect a pressure at a position where a pressure increase in the reactor 1 due to clogging of the heat exchanger 20 can be detected. The position of the pressure gauge 60 is preferably at an inlet of the raw material gas in the reactor 1 from the viewpoint of detecting a change in flow rate of the raw material gas to the reactor 1. However, the pressure gauge 60 may be provided at an arbitrary position inside the reaction tubes 1b and 1c, at an outlet of the reactor 1, inside the heat exchanger 20, a position between the heat exchanger 20 and the reactor 1, or the like.

In the embodiment of the present invention, a decrease in flow rate of the raw material gas to the reactor 1 is detected using the pressure gauge 60, but the detection device is not particularly limited so long as the device can detect the flow rate of the raw material gas to the reactor 1. For example, a flowmeter for detecting the flow rate of the raw material gas may be used in place of the pressure gauge 60, to provide the same effect.

The embodiment of the present invention allows: recovery of heat energy from the reaction gas; and prevention of reduction in, flow rate of the raw material gas to the reactor 1 due to clogging of the heat exchanger 20 and resulting reduction in production of (meth)acrylic acid.

The embodiment of the present invention may be easily applied to existing facilities because a simple structure of a by-pass tube 40 and a device for adjusting the flow rate of the reaction gas in the by-pass tube 40 allows: recovery of heat energy from the reaction gas; and prevention of reduction in production of products.

### EXAMPLES

### Example 1

Acrylic acid was produced through a vapor-phase catalytic oxidation reaction of propylene using the production apparatus shown in FIG. 1. The multitube reactor shown in FIG. 3 was used as the reactor 1.

A catalyst composed of a mixed oxide having an atomic ratio of Mo:Bi:Co:Ni:Fe:Na:Mg:B:K:Si=12:5:2:3:0.4:0.1:0.4:0.2:0.08:24 disclosed in JP 06-013096 B as an oxidation catalyst for oxidizing propylene to produce mainly acrolein was packed in reaction tubes of a first stage (hereinafter referred to as “first reactor”) of the multitube reactor.

On the other hand, a catalyst composed of a mixed oxide having an atomic ratio of Mo:V:Nb:Sb:Sn:Ni:Cu:Si=35:7:3:100:3:43:9:80 disclosed in JP 11-035519 A as a catalyst for oxidizing acrolein to produce acrylic acid was packed in reaction tubes of a second stage (hereinafter, referred to as “second reactor”) of the multitube reactor.

Liquefied propylene was passed through an evaporator and was supplied to the reactor 1 in a gas state as a raw material. Oxygen used in an oxidation reaction was supplied to the reactor 1 by pressurizing air with a compressor. Steam was supplied to the reactor 1 at the same time to avoid an explosive range of propylene. A raw material gas containing the above substances was supplied to the reactor 1 at the following fixed composition.

| | |
|---|---|
| propylene | 8.0 vol % |
| air | 68.6 vol % |
| steam | 23.4 vol % |

The first reactor packed with the catalyst for oxidizing propylene to produce mainly acrolein was operated at a heating medium temperature of 320° C. Further, the second reactor packed with the catalyst for oxidizing acrolein to produce acrylic acid was operated at a heating medium temperature of 260° C.

The reaction gas containing acrylic acid discharged from the reactor 1 was cooled to 150° C. by generating steam at 130° C. using the multitube heat exchanger 20, and was introduced into the absorption tower 30 for acrylic acid.

The absorption tower 30 for acrylic acid was provided with 50 baffle trays. Water as an absorbing liquid is sprayed toward the trays in the tower from the top of the tower, and acrylic acid in the reaction gas supplied to the absorption tower 30 is recovered as an aqueous solution from the bottom of the trays.

At the start of the operation, a pressure at an inlet of the reactor 1 was 60 kPa, but the heat exchanger 20 at an inlet of the absorption tower 30 was slightly clogged after 6 months. A pressure at an inlet of the reactor 1 increased to 70 kPa, causing difficulties in supply of raw material air. Thus, the composition of the raw material gas in the reactor 1 and the flow rate of the raw material gas to the reactor 1 were hardly maintained at constant values.

Then the valve 50 provided in the by-pass tube 40 of the heat exchanger 20 at an inlet of the absorption tower 30 was opened to adjust the pressure at an inlet of the first reactor 1 to 60 kPa. The raw material gas could be supplied at the initial composition and flow rate, thereby allowing continuous production operation of acrylic acid.

### INDUSTRIAL APPLICABILITY

According to the present invention, the use of the heat exchanger allows recovery of heat energy from the reaction gas, and the adjustment of the flow rate of the reaction gas bypassing the heat exchanger allows stable supply of the raw material gas even when a deposit adheres to the heat exchanger, to thereby enable stable and continuous production of (meth)acrylic acid.

According to the present invention, the adjustment of the flow rate of the raw material gas flowing through the by-pass tube to provide a substantially constant pressure of the raw material gas at an inlet of the reactor is more effective from the viewpoints of stable and continuous production of (meth)acrylic acid and prevention of reduction in productivity of (meth)acrylic acid.

1. An apparatus for producing (meth)acrylic acid comprising:
a reactor for producing (meth)acrylic acid through a vapor-phase catalytic oxidation reaction of one or two or more of propane, propylene, isobutylene, and (meth)acrolein in a raw material gas comprising one or two or more of propane, propylene, isobutylene, and (meth)acrolein, and oxygen;
a heat exchanger connected with the reactor, for cooling a reaction gas comprising the produced (meth)acrylic acid; and
an absorption tower connected with the heat exchanger, for contacting an absorbing liquid for absorbing (meth)acrylic acid and the reaction gas so that the (meth)acrylic acid in the reaction gas is absorbed into the absorbing liquid,
wherein the apparatus further comprises:
a by-pass tube for connecting the reactor and the absorption tower without interposition by the heat exchanger; and
a flow rate adjusting device for adjusting a flow rate of the reaction gas flowing through the by-pass tube.

2. The apparatus according to claim 1, wherein the flow rate adjusting device adjusts the flow rate of the reaction gas flowing through the by-pass tube to provide a substantially constant flow rate of the raw material gas to the reactor.

3. The apparatus according to claim 1, wherein the flow rate adjusting device adjusts the flow rate of the reaction gas flowing through the by-pass tube to provide a substantially constant pressure of the raw material gas at an inlet of the reactor.

4. A method for producing (meth)acrylic acid by recovering (meth)acrylic acid absorbed in an absorbing liquid, comprising the steps of:
generating (meth)acrylic acid by using a reactor through a vapor-phase catalytic oxidation reaction of one or two or more of propane, propylene, isobutylene, and (meth)acrolein in a raw material gas containing one or two or more of propane, propylene, isobutylene, and (meth)acrolein, and oxygen;
distributing a reaction gas containing the generated (meth)acrylic acid to a heat exchanger for cooling the reaction gas and to an absorption tower for contacting the reaction gas and an absorbing liquid for absorbing (meth)acrylic acid;
cooling the reaction gas supplied to the heat exchanger by using the heat exchanger; and
contacting the reaction gas cooled in the heat exchanger and the reaction gas distributed to the absorption tower in the distribution step in the absorption tower so that (meth)acrylic acid in the reaction gas is absorbed into the absorbing liquid,
wherein the reaction gas is distributed according to a flow rate of the raw material gas to the reactor in the distribution step.

5. The method according to claim 4, wherein the reaction gas is distributed to provide a substantially constant flow rate of the raw material gas to the reactor in the distribution step.

6. The method according to claim 4, wherein the reaction gas is distributed to provide a substantially constant pressure of the raw material gas at an inlet of the reactor in the distribution step.
